(19) United States
(12) Patent Application Publication
(10) Pub. No.: US 2003/0186353 A1
(43) Pub. Date: Oct. 2, 2003

(54) Microscopic precision construction of tissue array block

(76) Inventors: Page Erickson, Temecula, CA (US); Eugene Mechetner, Temecula, CA (US); Valeriy Katsnelson, Poway, CA (US); Norm Turoff, Poway, CA (US)

Correspondence Address: A. Justin Lum, Suite 128, 108 N. Ynez Ave., Monterey Park, CA, 91754, US

(21) Appl. No.: 10/402,864

(22) Filed: Mar. 29, 2003

Related U.S. Application Data
(60) Provisional application No. 60/369,618, filed on Apr. 2, 2002.

(51) Int. Cl. G01N 33/48; G01N 1/30; C12M 1/00

(52) U.S. Cl. 435/40.52; 435/283.1; 435/286.2; 435/287.3

(57) ABSTRACT

This present invention covers novel means, devices and instruments for production of a tissue array block that is further sectioned into duplicates of tissue arrays. An integral microscope is incorporated into the instant instrument for viewing and examining a stained reference slide and selecting donor tissue core region(s) from the reference slide. The said reference slide is held in a reference slide station that is operatively linked and indexed with a station or platform holding a source donor tissue block, which is further indexed and precisely positioned with reference to the donor punch needle for punching the donor tissue core(s). A recipient block indexed to the donor block punch is placed under the donor punch station and donor tissue cores are delivered into pre-existing hole(s) by a stylet to construct the tissue array block. The instant instrument comprises a donor punch station, optionally a second recipient punch station, with each operable independently or removable. The present invention also provides means of pre-loading needles with donor tissue cores for constructing tissue array blocks in pre-gridded and pre-punched recipient block. The tissue arrays produced from the tissue array blocks made according to the present invention are useful for testing such freshly-made and/or archival tissue specimens in both scientific and clinical research and applications.

### RELATED APPLICATION DATA

The present application claims priority under 35 U.S.C. § 119(e) to U.S. Provisional Application Serial No. 60/369,618 filed on Apr. 2, 2002, which is incorporated by reference herein.

### FIELD OF THE INVENTION

The present invention relates to precision instruments and devices for production of tissue arrays for analyzing biological specimens in the field of life sciences.

### BACKGROUND OF THE INVENTION

Fundamental understanding of biological, physiological and pathological processes and conditions often requires biochemical and histological analyses of multiple biological samples and specimens. With the advent of genome sequencing and proteomics technologies and the availability of whole genome sequences, gene sequences, and associated antibodies against gene products, massively parallel analysis of gene and protein expression and localization has become standard practice in biotechnology and biomedical research. As an example, tissue arrays (Kononen, et al., Nature Medicine, Vol. 4, No. 7, July, 1998) assembled from multiple tissue cores gridded on a single histological slide, provides tremendous technical advantage and economy relative to traditional immunohistochemical analysis of biological specimens, which are performed one sample per slide . For example, the utilization of these tissue arrays or “tissue chips” containing multiple miniaturized samples of tissue specimen have dramatically minimized the consumption of rare and limited specimen samples while simultaneously conserving valuable test reagents such as antibodies, enzymes, DNA and/or RNA reagents. This is because minute tissue samples sliced by microtome from tissue cores contain hundreds or thousands of cells, and these samples are adequate for most types of histological testing. Thus, tissue array technology becomes the preferred replacement technology over the traditional histochemical methodology, methodology that is tremendously wasteful of valuable biological samples and other reagent resources.

Tissue array technology is a major improvement in the means by which clinical pathologists and research scientists use tissue sections to analyze biological and pathological specimens and obtain critical information about the conditions and changes of the biological and pathological samples of interest. By utilizing tissue arrays, scientists have demonstratedsignificant improvements in the collection of information about cellular architecture, in-situ subcellular localization of gene and protein expression, and other issues relating to cellular function and processes. In practice, tissue array methodology large numbers of related or unrelated sample specimens fixed or gridded in a single array on a paraffin block. By sectioning the blocks of arrayed tissue cores with a microtome, hundreds of identical tissue array duplicates can be obtained from a single tissue array block, such that many different molecular and immunological tests may be performed on any particular array without exhausting the supply of tissue specimens. The miniscule amount of tissue used for each “tissue core” in a single microarray section means that a single fixed tissue (donor) block may be cored many times, so that tissue cores may be provided for making many tissue array blocks.

For example, instead of sectioning a tumor specimen into perhaps a hundred tissue sections, with each individual test being carried out on an individual single-slide section, the same tumor specimen can be made into cores to produce tens or even hundreds of tissue array blocks of the tumor specimen. Each of the tissue array blocks of the tumor specimen in turn may potentially be sectioned into six hundred tissue array slides containing the tumor specimen of interest, thereby increasing the supply of a testable tumor specimen by a factor of thousands, even for a small fixed tumor sample specimen. As compared to traditional histochemical methods utilizing one tissue section per test, this miniaturization and massively parallel analytical approach of tissue array technology provides a profound reduction in the labor and reagent costs of testing multiple tissue samples, while simultaneously enriching the information content of any tissue sample.

Tissue array technology also vastly expands the accessibility to precious tissue and pathological specimen libraries, thereby permitting laboratories unequipped for histochemical processing to conduct sophisticated histochemical testing. This new technical advance of tissue array is making both rare and common histological samples widely available for high throughput, large scale screening and testing of drugs, ligands and other biological interactions.

The manufacture of tissue arrays is conceptually straightforward. Individual tissue cores of 0.25 to 5 mm in diameter are punched from paraffin donor blocks containing individual histological specimens. The punched tissue cores are transferred to pre-gridded holes of equivalent diameter in a recipient block of paraffin or other suitable support matrix, to form a tissue array embedded in a paraffin block that is subsequently sectioned into hundreds of sections of a few microns thickness, with each section constitutes a multi-sample tissue array. In this way, a tissue core of just three millimeters in depth may be made into an individual tissue dot on about six hundred multi-sample tissue arrays.

The first successful attempt at developing an array of tissue specimens was by Battifora, et al. Lab. Invest. 55:244-248, 1986 and U.S. Pat. No. 4,820,504 “Multi-specimen tissue blocks and slides”. This type of array of tissues is commonly called the “sausage array”, wherein which cores of tissues are arrayed on a membrane. The membrane is rolled into a “sausage”, the rods fixed in place with embedding medium, and the sausage is sectioned to provide suitable microscopic sections for test purposes. Various claims are made for different forms of fixed and freeze dried tissues or other tissue samples. Separation of different types of tissue rods is claimed, in which groups of similar rods are isolated by use of a septum or septa. Such a method would therefore be useful for statistical sampling of specific tumor or tissue types, or if one were looking for a defining characteristic in a particular tissue type. However, the “sausage array” method is limited and notably lacks specific registration of the individual tissue rods. Not being able to unequivocally determine the position of each and every tissue rod means that the individual patient's clinical data or research sample history cannot be unequivocally related to any specific rod, because the rods have no specific address. Another deficiency of the “sausage array” methodology is that all phases of a block manufacturing are done manually by hand, a highly uncertain and inaccurate process. A similar effort in making a primitive array of tissues is presented by Furmanski, P., et al. U.S. Pat. No. 4,914,022, wherein the casing was improved using a paper straw encasing the rods.

Battifora and Mehta (In Lab Invest. 63:722-724, 1990 and U.S. Pat. No. 5,002,377, “Multi-specimen slides for immunohistologic procedures”) improved the above-mentioned original process by providing a multi-specimen tissue block of a spaced array with position registration. In the Battifora and Mehta process, prepared tissue samples are cut into a plurality of tissue strips. Multiple tissue strips are separated and grouped as desired, and positioned in parallel grooves in a mould. Embedding media is poured into the mould, providing a molded element comprising on one side a flat member and on the opposing side ridges containing individual tissue strips (square rods). Such molded elements are stacked, and the stack of elements are embedded in additional embedding media. Thus a block of embedded strips or rods are conjoined together in a spaced array. The spaced array provides registration indicative of the donor tissue source and the position of such sample sectioned from that such donor tissue in any section obtained from the array block. The Battifor and Mehta process, however, suffers the same manufacturing deficiencies and drawbacks of requiring manual handling and assembling that is both labor intensive and prone to errors. Clearly the Battifore and Mehta process is impractical for effective mass production of desired tissue array products.

What was lacking in the early days of generating tissue arrays was a precision instrument or machine to accurately provide and streamline tissue array manufacture, thereby reducing the cost and uncertainty of production. Recently, certain manual and semi-automated instruments, with certain patent rights granted, have become commercially available for the production of tissue arrays (Leighton B. U.S. Pat. Nos. 6,103,518 and 6,383,801, licensed to Beecher Instruments, Silver Springs, Md.). The Leighton instruments represent a significant advance in the art, providing the first effective method of machine fabrication for making tissue array blocks. These Leighton instruments, however, still suffer certain technical and conceptual shortcomings that interfere with efficiency, yield, and selectivity in the tissue array manufacturing process. These instruments, while adequate, can still be improved upon in order to efficiently produce consistent and uniform tissue arrays.

In the art of producing desirable tissue arrays, the most difficult tasks, and thus the foremost considerations, are (a) selecting and punching out desirable microscopic feature(s) from a donor tissue block; (b) delivering punched tissue cores efficiently and in perfect alignment into a punched hole in a recipient block for constructing a tissue array block; (c) constructing a tissue array block with matching depths of donor tissue cores with corresponding holes in the recipient block; and (d) embedding the donor cores at an even level with the surface of the recipient block, so that the donor tissue cores do not extend above the top surface of the recipient block with consequent risk of being dislodged from the hole when engaged by the microtome blade during sectioning. It is the primary objectives of the present invention and the instant instrument to overcome these issues.

For instance in the prior art, the tissue array instrument made by Beecher Industries (Leighton B. U.S. Pat. No. 6,103,518) indexes the donor and recipient punch needles by placing both punch needles on a single needle assembly carriage. The punch needles are brought into, and out of, alignment sequentially by a rotation of about thirty degrees in either clockwise or counterclockwise rotation. The entire process for punching and transferring donor tissue cores from the donor tissue block to recipient block is achieved by means of a single Z-axis and a single detent used by both the donor block sampling punch and the recipient block punch needles. The single detent is normally set for punching the recipient block holes. This device provides that (1) the donor tissue block and recipient block must share a common Z-axis and a common Z-axis detent; (2) the common Z-axis detent is set for the position for punching the recipient block, while the donor tissue block is presented to the punch needle by means of a “bridge” that fits over the recipient block mount; and so that (3) the donor punch needle will engage the donor tissue block, often achieved with different depths or variable thickness, at a substantially different level as compared to the recipient block, thus, requiring best-educated manually controlled depth estimation, (4) the internal “stylus” that pushes the donor tissue core is longer than the donor punch needle, which also requires best-educated manually controlled depth estimation when delivering the tissue core into the recipient block. This device and practice so performed using the device will not provide donor tissue cores of exactly the same top surface height as the top of the holes in the recipient block, thus, resulting in certain numbers of donor tissue cores protruding above or falling below the top surface of the recipient block. The tissue array block so constructed with an uneven top surface will often suffer damage when being sectioned by the microtome, wherein the donor tissue cores or plugs protruding from the top surface may be pulled out from the tissue array block or, in the case where donor tissue cores or plugs below the top surface, the initial batch of tissue array sections will lack representation of certain donor tissue cores.

In an improved instrument made by Beecher Industries (Leighton B. U.S. Pat. No. 6,383,801), the donor and recipient punch needles are assembled on two separate punch arms, which are parallel to one another and are moveable along a Z-axis by pneumatic or hydraulic drives that provides continuous movement controlled by a computer. The improved instrument, however, still does not address the issue of indexing the donor tissue block to the reference slide by operatively linking the step of microscopic viewing and examining with that of the donor tissue core punching and other issues posted by the prior Beecher instrument as discussed above. The new instrument employs hydraulic or pneumatic drives for the punch needle assemblies with Z-axis movement controlled by a computer, which controls X- and Y-axis movement for all other platforms, thus, increasing the costs of the instrument dramatically.

### SUMMARY OF THE INVENTION

The instant instrument overcomes the aforementioned deficiencies by many unique designs of the present invention. As aforementioned, one of the most inconvenient and thus, inefficient processes in constructing a tissue array block is the selection of tissue cores from donor tissue blocks. Each donor tissue embedded in paraffin media in a donor tissue block needs to have a reference donor tissue slide sectioned from the top surface of that donor tissue block. Then specific areas of interest of the donor block tissue sample are selected by examining the reference slide under the microscope, necessitating some means by which the corresponding region on the actual donor block can be selected and cored. This selection should be highly accurate, since tissue cores may be 0.5 mm or less in circumference. To accomplish this, the reference donor tissue slide is histologically and/or immunohistologically stained to reveal the cellular and subcellular features of the donor tissue under microscopic examination. The desired region(s) or tissue cores are selected and marked correspondingly on the donor tissue block for punching and withdrawing the desired tissue cores for further construction into a recipient paraffin block having pre-drilled holes fitting snugly for the donor tissue cores. As commonly practiced using the Leighton's instruments, the reference donor tissue slide(s) is examined under a stand alone microscope and the desirable tissue core region(s) marked and realigned onto the donor tissue block, often an overlaying film or transparent copy over the top of the donor tissue block for marking and indexing purpose. Since the microscopy and the individual donor tissue blocks are not linked or indexed, the process of microscopic examination for tissue core regions and subsequent alignment and marking of the donor tissue block is very labor intensive and cumbersome and not opted for processing very large number of donor tissue blocks.

It is the primary objective of the present invention to provide easy and accurate selection and punching of donor tissue cores by operatively linking the microscopic examination step with the alignment of the punching unit precisely onto the selected donor tissue core regions in the donor tissue block. This objective is achieved in the present invention by operatively linking a reference slide station (hereinafter as Reference Slide Station) with a donor tissue block station (hereinafter as Donor Block Station). The Reference Slide Station has a holding device for holding a stained reference slide and is situated conveniently under a microscope for microscopic examination. The Donor Block Station has a holding device for holding at least one donor tissue block and is movable along the X- and Y-directions, or left-right, and forward-backward movement, respectively. The Donor Block Station has a range of movement so that it can be stationed directly underneath either the microscopic viewing area for indexing purpose against reference points on the donor reference slide or the donor punching unit for punching donor tissue core(s). Both the Reference Slide Station and the Donor Block Station are situated on a common platform, and are thus operatively linked and indexed against one another.

In a preferred embodiment, the instant instrument is constructed on a base plate that has a leveling device (the circle with water at the front of the machine) and an adjustment mechanism to level the machine.

The microscope, with an adjustable light source enhancing the viewing field, of the present invention has a “center ocular reticule,” with the cross center of the reticule pinpointing to the center of the donor tissue core region. The microscope is stationary and the Reference Slide Station can be adjusted so that the ocular center cross of the microscope reticule pinpoints to the middle of a tissue core region, which is indexed precisely to place the tissue core region of the donor tissue block to be directly under the stationery punch needle on the Donor Punch Needle Station. The binocular head of the microscope can also be adjusted in the X and Y directions to center the ocular reticule to pinpoint the center of the donor tissue core region. The reference slide is so positioned in spatial relationship with the donor tissue block so that reference slide is synchronized with the donor tissue block so that the center cross of the reticule pinpoints to the point on the reference slide and the donor punch needle pinpoints the corresponding region on the donor tissue block. The instant instrument has means and devices by the operatively linked Reference Slide Station and Donor Block Station so when punching the donor tissue cores, the middle of the ocular cross of the reticule will correspond to the middle of any donor tissue core punched out of the donor block by the donor punch needle. In summary, the Reference Slide Holder and Donor Block Holder, and thus the Donor Punch Needle, are synchronized. Once the operator sets the index of the reference slide to correspond to the donor tissue block held in the donor block holder, moving the reference slide holder under the microscope by moving the common Slide/Donor Platform will also move the donor tissue block so that the same core region in the donor tissue block will be punched by the donor punch needle.

The present invention of operatively linking the Donor Block Station with the Reference Slide Station offers a tremendous advantage over the methods used in the prior art, wherein the donor block is stationed independent of microscopic examination, whereby intensive human maneuvering and guesswork are involved to view and examine the reference slide(s), mark the reference points on an overlay, and place the overlay with the marked reference points onto the donor tissue block. It is time-consuming and inaccurate, especially when hundreds and thousands of reference slides and donor tissue blocks need to be examined and indexed manually. The present invention overcomes the shortfalls of the prior art by linking the microscopic examination and selection of the tissue core regions with the indexing of the donor tissue blocks, thus simplifying and perfecting a precision selection process for identifying specific donor tissue core regions and punching out the selected donor tissue cores with high accuracy.

One of the problems encountered during the punching and withdrawing of donor tissue cores is the breaking off of the tissue cores at the bottom of the punching needle. In the first attempt, the Beecher instrument made by Leighton (U.S. Pat. No. 6,103,518) provides a punch needle assembly with a stylet extending beyond the end of the needle. The purpose of this type of design, controlled by electrical contacting of the punching needle and the stylet, is to provide a current signal when the stylet touches the punching needle, to indicate that the donor tissue core has been expelled from the punch needle. However, a stylet that extends beyond the end of the punch needle can compress and damage both the donor tissue core and the recipient array block. Damage is particularly likely if the donor tissue core is already too long by virtue of the inconsistent donor core cutting process. Recently, Leighton (U.S. Pat. No. 6,383,801 B1) through Beecher offers a pneumatic or hydraulic cylinder for the punching unit wherein a controlling force of tamping is applied to the stylet so subtle friction griping of the tissue cores would aid the withdraw of the punching needle from the donor tissue block and breaking off the tissue core at the bottom of the needle. This type device is very sophisticated and complex, thus, the punching unit of outer hollow needle and inner stylet has to be reusable and not disposable. The present invention provides a simple yet elegant design that employs a rotatable needle collet (FIG. 8) to hold a disposable punch needle. When turned or rotated by means of a handle (FIG. 8, item 6), the punched tissue core will be broken at the bottom of the punch needle thus freeing the punch needle and the donor tissue core from the donor tissue sample. A simple rotatable needle collet of the present invention provides efficient and precise break off of the donor tissue core at the bottom of the punch needle, thus making possible the punch needle-stylet assembly as a disposable item. With a disposable punch needle-stylet assembly, cross contamination of tissues and tissue cells are avoided when such a reusable punch needle-stylet assembly is used.

Another problem in constructing a tissue array in a recipient block is the punching holes in the block in a regular grid pattern. In the prior art, a recipient block is situated on a platform that positions the recipient block under a recipient punch unit for punching holes. The platform holding the recipient paraffin block needs to be constantly adjusted for positions along the X- and Y-axis by linear drives between the step of punching the hole and the step of receiving the donor tissue core. The common practice of sequential steps of punching holes in the recipient block followed by receiving donor tissue cores require two separately operable punch units with two parallel Z-axis and constant adjustments of the recipient block in between the donor punch unit and the recipient punch unit for punching holes and receiving tissue cores, respectively. Perfect alignments of positions are required or any mis-alignment would cause damage to the donor tissue cores and the recipient array block. The present invention provides a pre-gridded recipient array block with holes pre-punched in a regular pattern of pre-determined distance and space; This is accomplished by detents incorporated into the X-axis and Y-axis drives. Thus the holder for the recipient array block can move to the pre-determined stops or detents of both X- and Y-axis in a regular pattern. Upon indexing and fixing the first hole position of the recipient block, the rest of the holes in the recipient block are indexed by the pre-set detents or stops for receiving the tissue cores. The utilization of pre-made and pre-gridded recipient paraffin block takes out the constant guess work and manual maneuverings that cause misalignment and damage to the tissue cores and recipient block. The supply of pre-made recipient blocks, with holes punched out, also eliminates the need for a recipient punch unit so that a single donor punch unit with pre-loaded disposable punch needle-stylet assemblies with pre-loaded tissue cores will effectively provide means to deliver donor tissue cores into the holes of the pre-made recipient block.

In the prior art as disclosed by the Beecher instrument, the movements along the X-axis and/or Y-axis are adjusted continuously by using two separate micrometered drives, such as pneumatic or hydraulic drives, one along the X-axis and one along the Y-axis. The use of the micrometered drives in the Beecher instrument requires that each hole location in the recipient array block to be “dialed in” or calculated in for both the X-axis and Y-axis, a tedious, non-precision, rate limiting step that substantially slows down the process of constructing tissue array blocks.

In a preferred embodiment, the instant instrument employs a knob-shaft assembly for X- and Y-axis movements so that, when actuated, the shaft turns a specified number of degrees before engaging a new detent. For example, each X- and/or Y-movement of the Recipient Block Station engages in one detent stop providing a perfected grid position for punching out a hole by the Recipient Punch Needle. Once the first hole in the grid is created and indexed to the donor punch needle, any subsequent donor tissue core punched out by the donor punch needle can be delivered directly to the next hole in the grid by moving the recipient array block the appropriate number of detent stops prior to moving it under the Donor Punch Needle Station.

The present instrument and different variations of the instant instrument offer many major advances and improvements over the prior art. To summarize, the instant instrument and many variations thereof comprise, among many other unique features, an operatively linked Microscope, with Ocular Reticule, to view, examine, align and index the Reference Donor Slide with the Donor Tissue Block for the purpose of precisely selecting tissue features and capturing them in cores through use of the Donor Needle Punch; a separate holder for a Stained Reference Slide (sectioned from the Donor Tissue Block and stained) operatively linked and indexed to the Donor Tissue Block for precision punches; separate precision holders for the Donor Tissue Block (under Donor Punch Needle Unit) and a Recipient Block (under Recipient Punch Needle Unit); the Recipient Block indexed to the Donor tissue Block; precision-indexed platform(s) to move the Recipient Block, the Donor Tissue Block, and the Reference Slide in perfect alignments in reference to one another; precisely pitched stops or detents on the holder of the Recipient Block to exactly position holes for receiving the Donor Tissue Cores for constructing the Tissue Arrays; independent Needle/Stylus Assemblies that are disposable wherein the Needle and Stylus are of the same length for precise “Z” Height delivery of donor tissue core into Recipient Block, the said “Z” Height stops are adjustable for the separately operable (1) Donor Needle-Donor Block interface, (2) Recipient Block to interface with Donor Needle, and (3) Recipient Needle to punch Recipient Block; the said Needle/Stylus Assemblies are easily assembled in a rotatable Collet device for quick needle changes and breaking off tissue cores by rotation; independent or detachable Recipient Needle Station with second detent for use with disposable needles; independent Donor and Recipient Needle Stations; disposable Needles and disposable Needles pre-loaded with cores for custom array assembly; and pre-made pre-gridded recipient block.

### BRIEF DESCRIPTION OF THE FIGURES

FIG. 1A. is an isometric and semi-schematic drawing of the present invention, seen from the operator's perspective, showing Slide Holder, Slide/Donor Platform, Donor Block Holder, Recipient/Donor Platform, Deck Plate, Recipient Stage, Recipient Block Holder, Donor Needle Station and Microscopy Station;

FIG. 1B. is a schematic drawing of the present invention, showing the Slide/Donor Platform moved left ward that places the Donor Block Holder under the Microscopic viewing field in the Microscope Station;

FIG. 1C. is a schematic drawing of the present invention, showing the Slide/Donor Platform movement of returning to its original position from left moving to right, with the dotted lines showing the transition from left to right position;

FIG. 2A. is a semi-schematic drawing of the present invention and instant instrument, seen from the operator's perspective, showing Slide Holder, Slide/Donor Platform, Donor Block Holder, Recipient/Donor Platform, Deck Plate, recipient Stage, Recipient Block Holder, Recipient Needle Station, Donor Needle Station and Microscopy Station; and

FIG. 2B. is same semi-schematic drawing of the present invention and instant instrument, seen from the operator's perspective, showing X Handwheel, Y Handwheel, recipient Y Detent Wheel, Thumbwheel, Recipient X Detent Wheel, Stylus and Needle Adjustment Knobs.

FIG. 3 is an enlarged and isometric view of the Slide Holder of the present invention, showing holding a slide, as shown in FIG. 2A

FIG. 4 is an enlarged and isometric view of the Slide/Donor Platform of the present invention as shown in FIG. 2A.

FIG. 5 is an enlarged, isometric and detailed view of the Recipient/Donor Platform of the present invention as shown in FIG. 2A.

FIG. 6 is an enlarged, isometric and detailed view of the platform subassembly of the Recipient/Donor Platform of the present invention as shown in FIG. 2A and FIG. 5.

FIG. 7 is an isometric semi-schematic drawing of the Donor Needle Station of the present invention as shown in FIG. 2A.

FIG. 8 is an exploded isometric and detailed view of a Punch Needle-Stylus assembly of the present invention with a rotatable Collet rotatable by the handle.

FIG. 9 is an isometric and detailed view of a needle of the present invention.

FIG. 10 is an enlarged view of the microscopic reticule of the present invention viewing and examining the desirable cellular or subcellular features on the stained reference donor tissue slide for selecting desirable donor tissue regions.

FIG 10B is an enlarged view of the microscopic reticule of the present invention viewing and examining the tissue sample of a donor tissue block showing where a donor tissue core has been removed.

FIG. 11A is a cross-sectional view of a donor tissue block of the present invention with and without a donor tissue core removed.

FIG. 11B is a cross-sectional view of a recipient paraffin block of the present invention, empty and filled with donor tissue core.

### DETAILED DESCRIPTION OF THE INVENTION

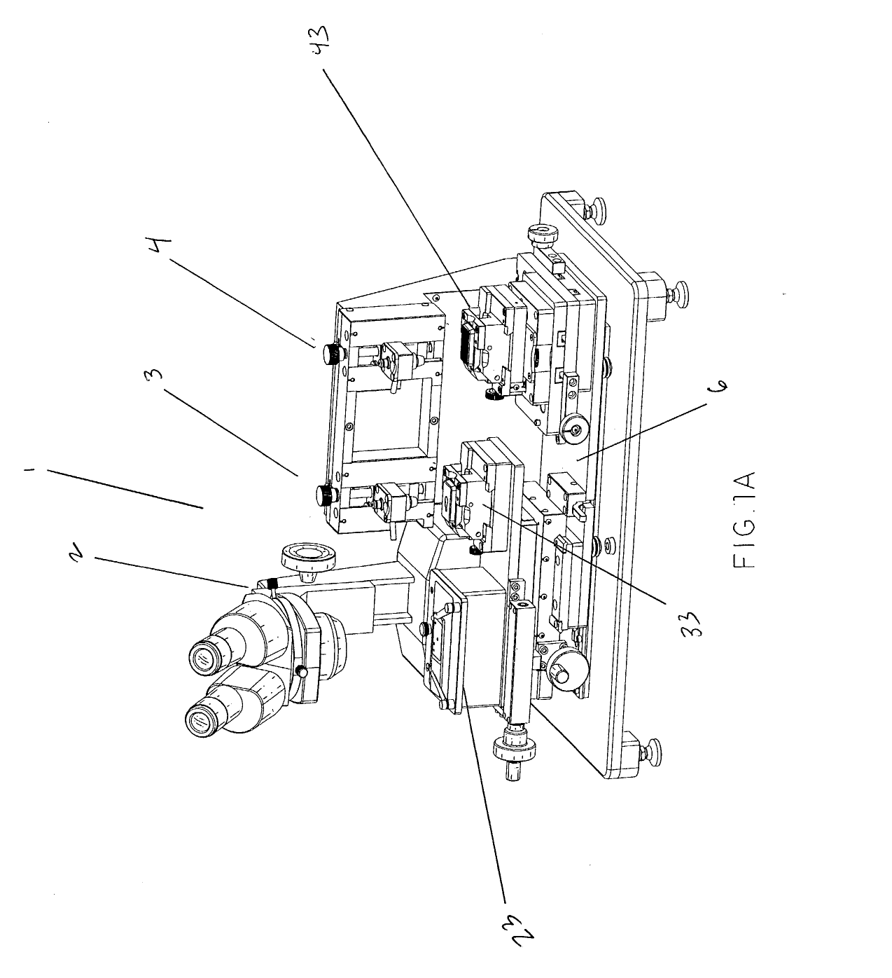

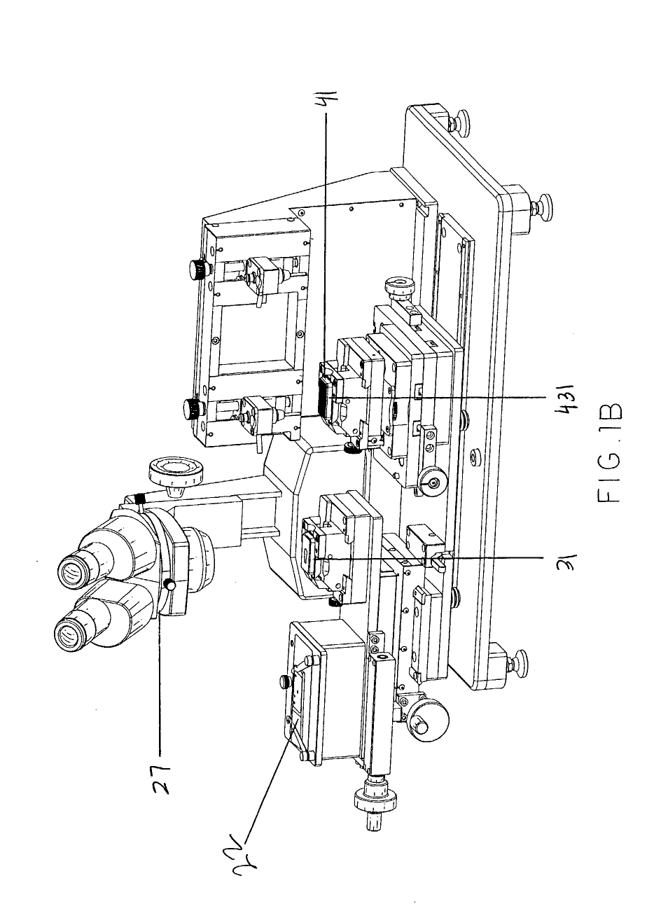

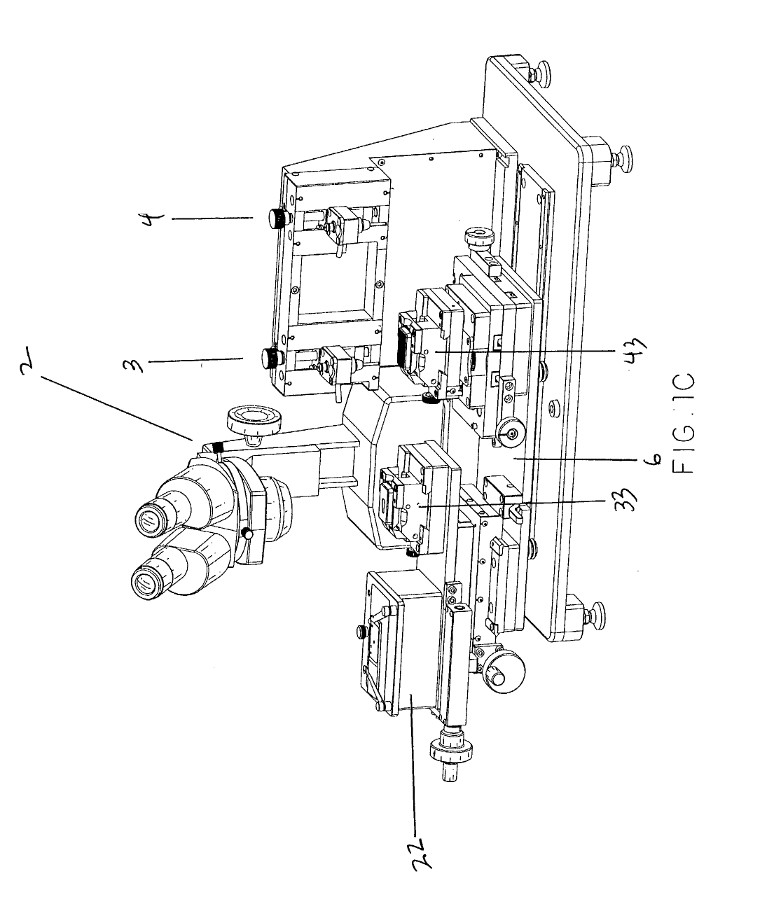

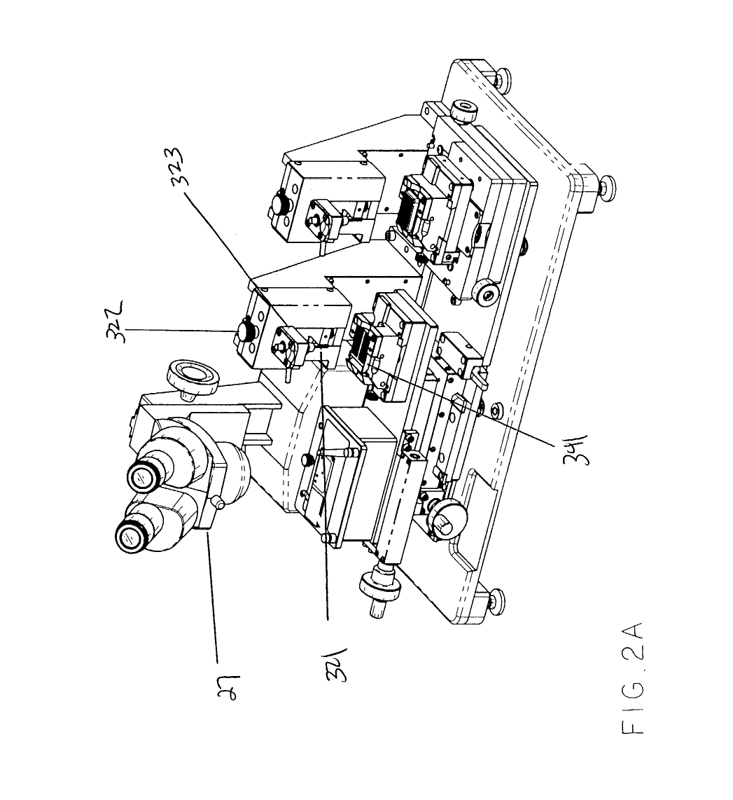

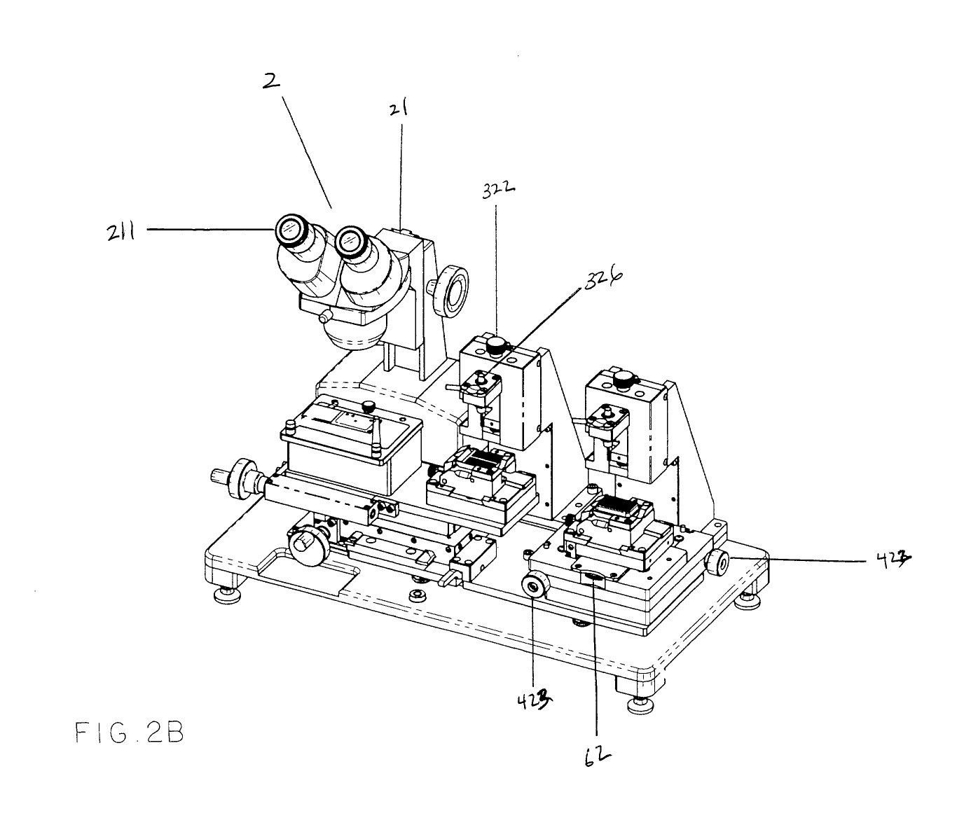

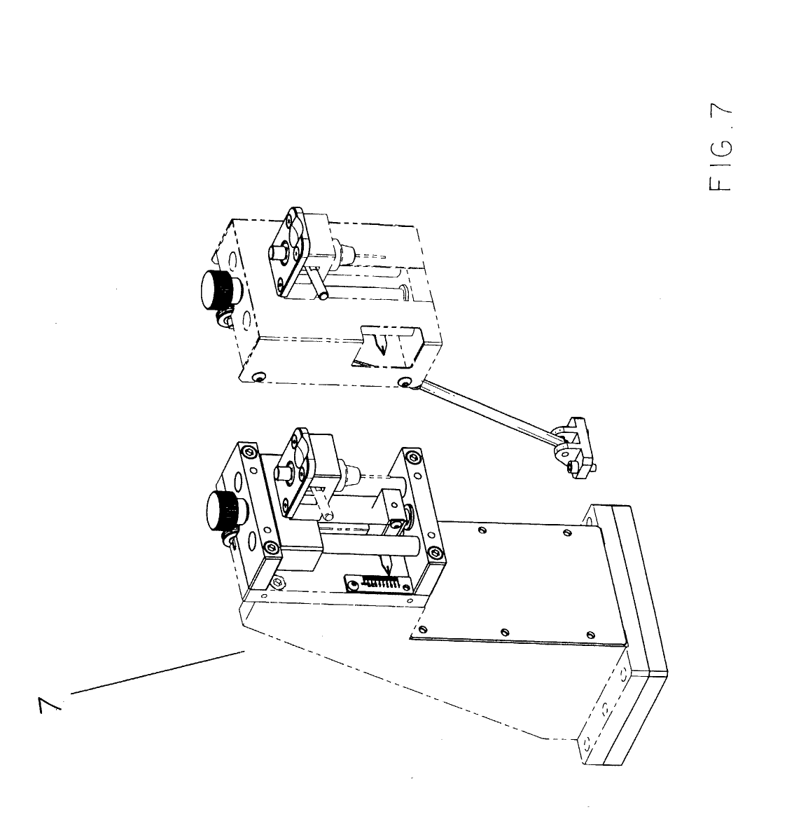

As a primary objective, the present invention 1, as illustrated in by a preferred embodiment in FIG. 1, operatively links the step of microscopic reviewing and examining the stained donor reference slide 22 and the selection of the donor tissue core 312 regions by indexing the donor tissue block 31 to the punching-out of the donor tissue cores 312 from a donor tissue sample 311 of the donor tissue block 31 by a donor punch needle-stylus assembly 32. In a preferred embodiment of the present invention, the instant instrument 1 directly links and places specific microscopic features of a fixed tissue sample 311 in the donor tissue block 31 into the vertical path of the donor needle punch 321 in the donor needle station 3 as illustrated in FIG. 2A and FIG. 7, such that when the donor needle punch 321 is operated, the selected tissue feature will be obtained in a punched donor tissue core 312. The punched donor tissue core 312 may then be subsequently transferred to a receptacle or hole previously punched in a paraffin recipient paraffin block 41. As illustrated in FIG. 2A, a novel feature of the instant instrument 1 is the provision of a microscope station 2 being incorporated into the instrument 1 and a reticule adjustment mechanism 27 to index the microscopic field 28 precisely to the donor tissue block 31 by operatively linking a Reference slide holder 23 holding the reference slide 22 with a donor block holder 33 holding a donor tissue block 31 by means of a common slide/donor platform 5. The present invention thus synchronizes the step of microscopic viewing, examination and selection of donor tissue core 312 region(s) with that of punching donor tissue cores 312 from the donor tissue block 31 through indexing microscopic features onto the donor tissue block 31 via operatively linking the reference slide holder 23 and the Donor Block Holder 33 on the common slide/donor platform 5.

As shown in FIG. 2A, in a preferred first operation and embodiment, the donor tissue block 31, which is a paraffin block containing a fixed tissue sample specimen 311 is locked into place on a donor block mechanism 34. Subsequently, a first stationary donor needle punch 321 is adjusted maximally to a depth sufficient to just punch through the donor tissue block 31 without contacting a standard cassette 341 of the donor block mechanism 34. The depth is typically determined by depressing the finger plate 323 of the donor punch needle stylus assembly 32 just until the needle 321 of the assembly 32, when placed alongside the donor tissue block 31, just touches the cassette 341 itself and then is fixed in place at a set height no longer than the depth to touch the cassette 341 by a donor needle adjustment knob 322 on the donor needle station 3. As seen in FIG. 2B, when the donor needle punch stylus assembly 32 is operated against the donor tissue block 31, the donor needle punch 321 will extend to the depth of thickness of the donor tissue block 31. The Z-axis travel of the donor needle punch 321 is stopped or detented at this depth by means of the donor needle adjustment knob 322 on the Donor Needle Station 3. This will punch and retrieve a donor tissue core 312 of just the thickness of the source donor tissue block 31 when the donor needle punch 321 is fully depressed to its pre-set full stop or detent. In this preferred embodiment, the present invention 1 provides a unique feature of accommodating tissue donor blocks of varying heights by instantaneous measurement of donor block depth and adjusting the punching depth accordingly. The donor block mechanism 34 has a quick release 342 for making X- and/or Y-axis adjustments of the donor block holder 33.

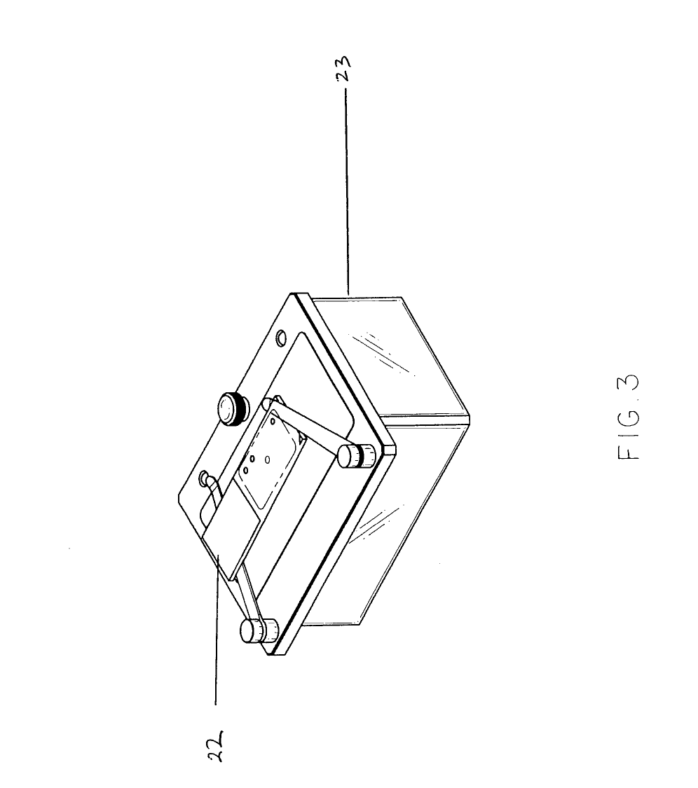

As shown in FIGS. 2A and 3 of the preferred embodiment of the instant instrument 1 of the present invention, the microscope station 2 of the instant instrument 1 provides a magnified visual viewing field for inspecting both the donor tissue block 31 surface and the stained reference slide 22 sectioned off the top of the source donor tissue block 31, wherein said stained reference slide 22 is mounted on the reference slide holder 23. The stained reference slide 22 is prepared from a full section taken from the source donor tissue block 31 and thus is entirely representative of all features of the top of the donor tissue block 31. For visualization and easy microscopic viewing, the reference slide 22 is developed by appropriate histochemical staining (such as hemotoxylin-eosin staining). A cross-line reticule 24 is provided in the microscope 21 eyepiece 211 for precise localization and selection of any desired features in the microscopic visual field, as well as a light source 26 that is mounted on the Microscope 21 to enhance visual viewing and inspection of the microscopic field 28.

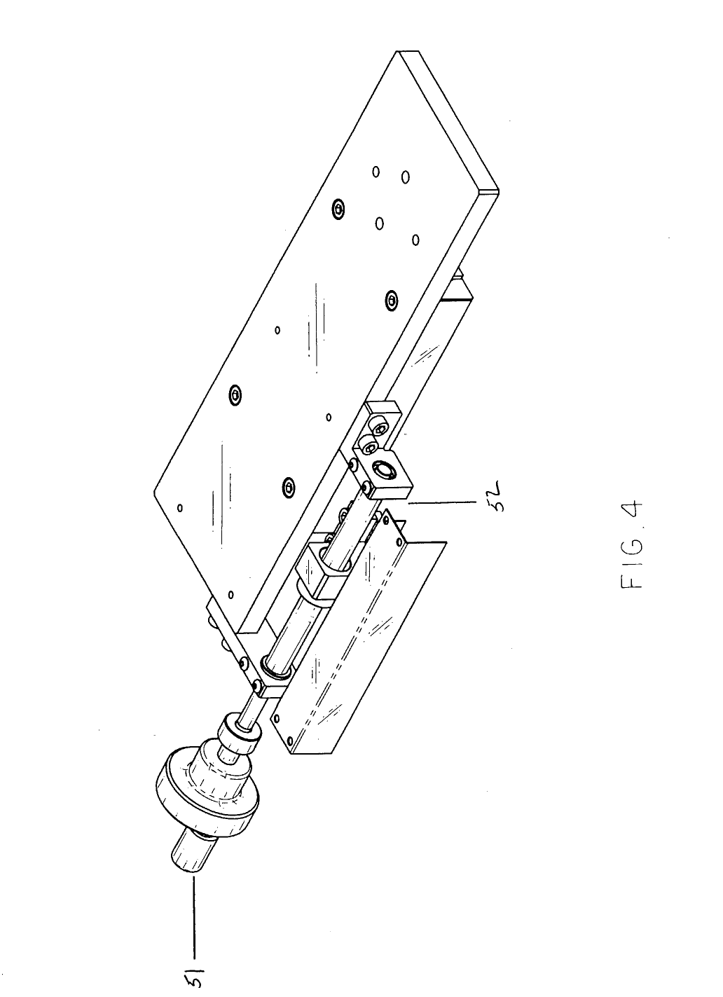

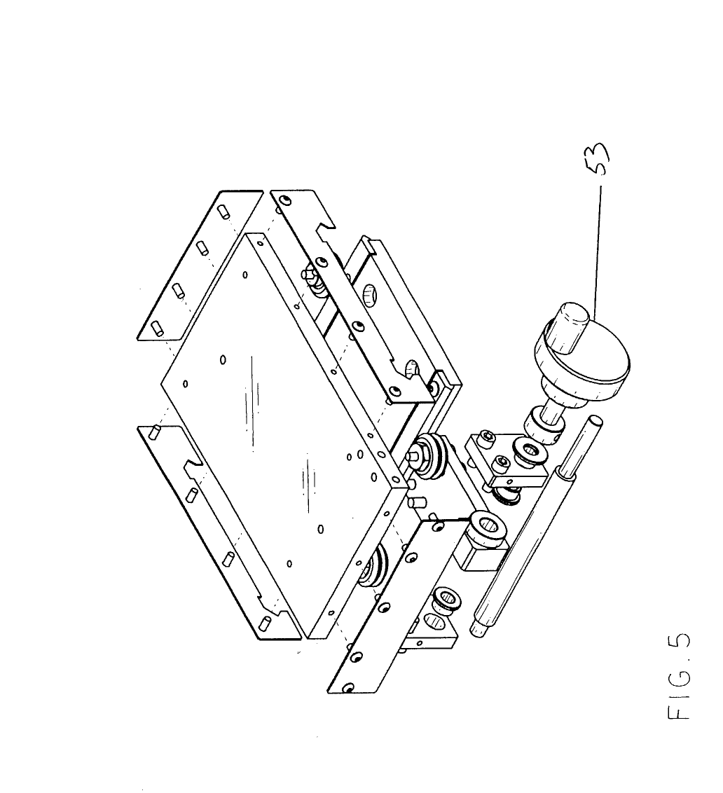

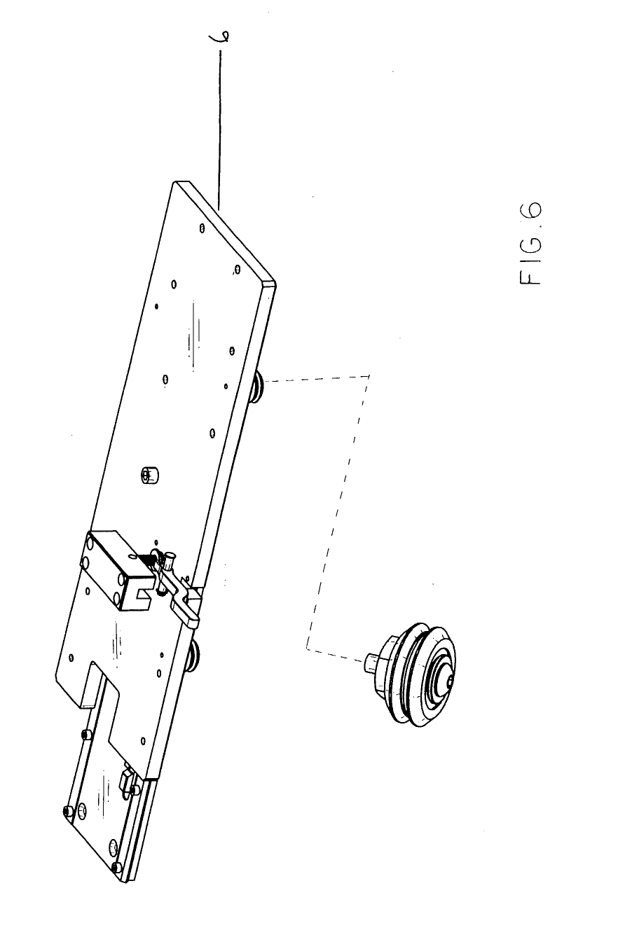

As shown in FIG. 2B, FIG. 4 and FIG. 5, in a preferred second operation and embodiment, a desired feature (such as an anomalous margin of the specimen) is selected on the donor tissue block 31 by visual inspection of the stained reference slide 22. The reference slide holder 23 and the donor block holder 33 are operatively linked and both fixed onto the common slide/donor platform 5 and operated in sync along the X-axis and Y-axis by means of an X handwheel 51 and Y handwheel 53. The slide/donor platform 5 is provided with a slide mechanism 52 with detents and provides a means by which the slide/donor platform 5 can be moved along the X-axis so that the reference slide holder 23 and reference slide 22 can be moved out (left movement) from under the stationery microscope 21, whereupon the donor tissue block 31 is simultaneously positioned under the microscope 21. The process may be reversed by moving the slide/donor platform 5 to the right, returning the reference slide holder 23 under the microscope 21 and the donor tissue block 31 back under the donor needle station 3. The slide/donor platform 5 is precisely and conveniently movable along the X- and Y-axis by means of the handwheels 51, 53, such that the viewing field of the microscope 21 is smoothly switched onto either the stained reference slide 22 or the top of donor tissue block 31 for indexing purposes. Using the handwheels 51, 53, the selected desired regional feature of the donor tissue block 31 can be moved directly under the donor needle punch 321. The slide/donor platform is iteratively moved in left and right directions so that the donor tissue block 31 is indexed directly under the reticule 24 of the microscope 21 in exact reference to the selected desired features on the stained reference slide 22. Fine-tuned and precision adjustments using the handwheels 51, 53 on the slide/donor platform 5 provide perfect alignment of the selected desired feature directly under the donor needle punch 321. As shown in FIG. 2A, upon completion of the described indexing operation, any movement of the slide/donor platform 5, which is actuated by the X- and Y-axis handwheels 51, 53, will be perfectly indexed between the stained reference slide 22 and the donor tissue block 31, such that any feature on the reference slide 22 directly under the reticule 24 will be positioned directly transecting the Z-axis of the donor needle punch 321 in the donor needle station 3.

In yet a preferred third operation and embodiment, the recipient paraffin block 41 is moved from under the recipient needle station 4 by releasing the latch 61 on the recipient/donor platform 6. As shown in FIG. 2B, the recipient paraffin block 41 is moved to the left to be directly under the donor needle station 3. By using the thumbwheel 62, the upper surface of the recipient paraffin block 41 is just mated to the fully depressed donor needle punch 321. The donor needle punch 321 is released and the recipient paraffin block 41 is returned to be directly under the recipient needle station 4. In this maneuvering, the donor needle punch 321, without the need for an additional detent on the movement of the donor needle punch 321, is indexed to the correct height for the delivery of donor tissue cores 312 to the recipient paraffin block 41 to accommodate the different heights at which cores are obtained and dispensed between the donor tissue block 31 and the recipient paraffin block 41.

In a preferred embodiment, the donor needle station 3 and recipient needle station 4 are synchronized as well, so that the donor tissue core 312 will be deposited in a proper recipient hole 411 when the recipient paraffin block 41 is moved under donor punch needle station to receive the donor tissue core 312.

As shown in FIG. 2B, to core or drill holes in the recipient paraffin block 41, a recipient needle punch 42 is adjusted, when placed alongside the margin of the recipient paraffin block 41, by depressing it fully until it just touches the recipient cassette 431 of the recipient block mechanism 43. The recipient paraffin block 41 is then centered approximately under the Z-axis of the recipient needle punch 42 using the X and Y Detent Wheels 422, 423, such that the detents of both the X and Y linear drives of the recipient block mechanism 42 are engaged. By selection of a specific grid pattern for the final array, the number of X and Y detent steps are selected which are appropriate to move the recipient paraffin block 41 to the location of the first hole in the selected grid pattern. The recipient needle punch 31 is depressed, and rotated by the recipient needle collet 44 to break off a core from the recipient paraffin block 41, retracted, and the recipient stylus button 45 depressed. The paraffin core is ejected and discarded. This maneuvering step may be repeated until the grid is complete, or optionally, the recipient paraffin block 41 hole formation may be cycled along with donor tissue block 31 core formation.

In a preferred embodiment, the recipient block mechanism 43 of the instant instrument employs means of a knob-shaft assembly 46 for X- and Y-axis movements so that, when actuated, the shaft of the recipient block mechanism 43 turns a specified number of degrees before engaging a new detent. For example, each X- and/or Y-movement of the recipient block mechanism 43 engages in one detent stop for a perfect grid position for punching out a hole by the recipient punch needle 42. Once the first hole in the grid is created and indexed to the donor punch needle 31, any subsequent donor tissue core 312 punched out by the donor punch needle 31 can be delivered directly to the next hole in the grid by moving the recipient paraffin block 41 the appropriate number of detent stops prior to moving it under the donor needle punch 321.

In a preferred embodiment of the present invention and instant instrument, there are three types of Z-height adjustments for the donor punch needle stylus assembly 32 and the recipient punch needle stylus assembly 47. The following lists the importance of each adjustment and order of adjustment:

(a) donor punch adjustment is performed so that the donor punch needle 321 can push into the donor tissue block 31 just above the standard block cassette 341 under the donor tissue block 31 without touching the cassette 341.

(b) recipient block adjustment is performed so that the top of the recipient paraffin block 41 is at the same level as the end of the donor punch needle 321 stationed above the donor tissue block 31.

(c) recipient punch needle adjustment is performed so that the recipient punch needle 42 will go down into the recipient paraffin block 41 just above the standard cassette 341 under the recipient paraffin block 42.

In a preferred embodiment, the Recipient Block Station 7 has its own X and Y-axis adjustment mechanisms 71, 72 to set pitch based on the recipient punch needle 42 sizes. The instant instrument currently provides four recipient needle sizes that are commonly used and the pitch movement is set or moved by turning knobs a certain number of clicks, with each of 1-4 clicks corresponding to the size of recipient punch needle in use: the greater number of clicks, the larger the size of the recipient punch needle 42.

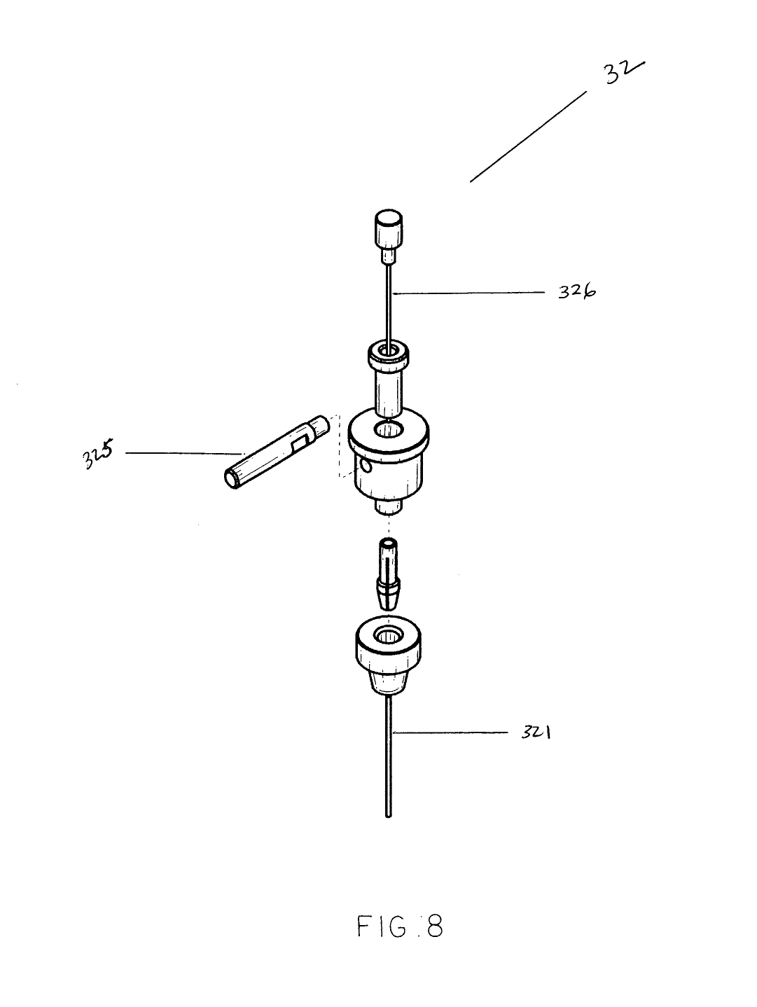

As show in FIG. 8, in a further preferred operation and embodiment, the donor needle punch 321 is depressed to its full displacement into the donor tissue block 31, the needle collet 324 rotated by means of a handle 325 to break off the core free, and retracted. A donor tissue core 312 is now present in the donor needle punch 31, which has an internal diameter equal to (or slightly smaller than) the outer diameter of the recipient needle punch 42. The latch 61 on the Recipient/Donor Platform 6 is again released, the Recipient/Donor Platform 6 is moved so that the recipient paraffin block 41 is moved under the donor needle punch 321, and by virtue of the factory-set indexing between the donor needle punch 321 and the recipient needle punch 42, the donor needle punch 321 is directly over the hole selected previously by use of the X and Y Detent Wheels 73, 74.

The donor needle punch 321 stylus 326 is depressed, by which means the donor tissue core 312 is extruded into the selected recipient hole 411 in the recipient paraffin block 41. The donor needle punch 321 is released, being drawn up from the recipient paraffin block 41 by a spring in the donor punch needle stylus assembly 32 and the recipient paraffin block 41 is returned to its position under the recipient needle station 4 by releasing the detent latch on the recipient/donor platform 6.

It is understood that all movements of the holders under the several stations of the instant instrument are terminated and stabilized by factory adjusted detents on the slide/donor platform 5 and recipient/donor platform 6.

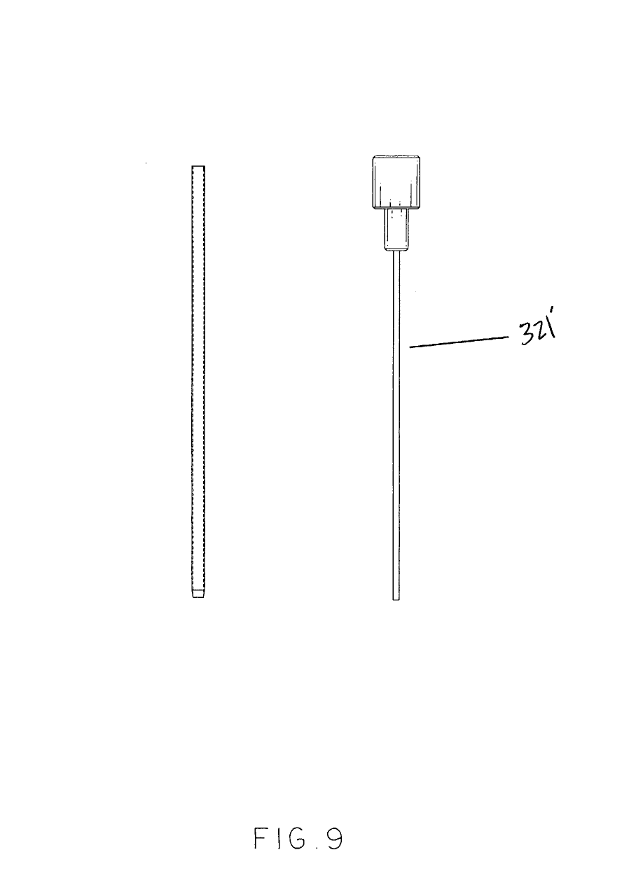

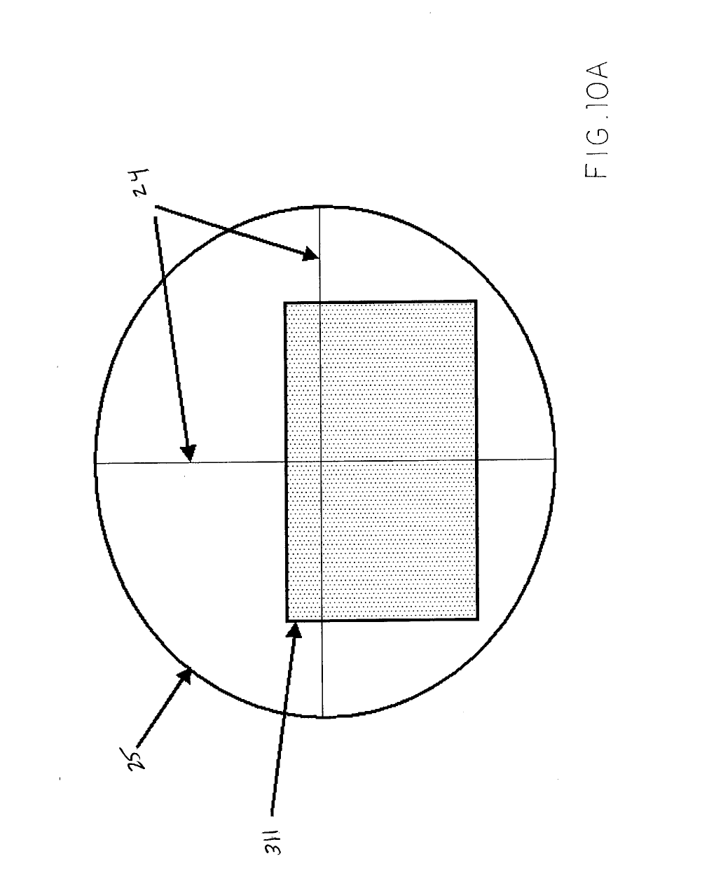

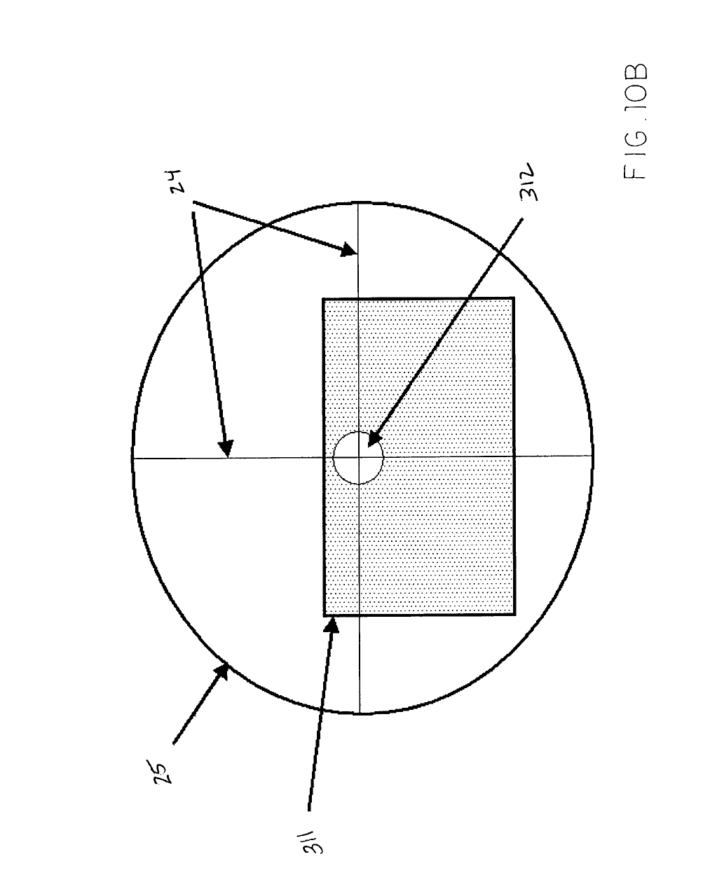

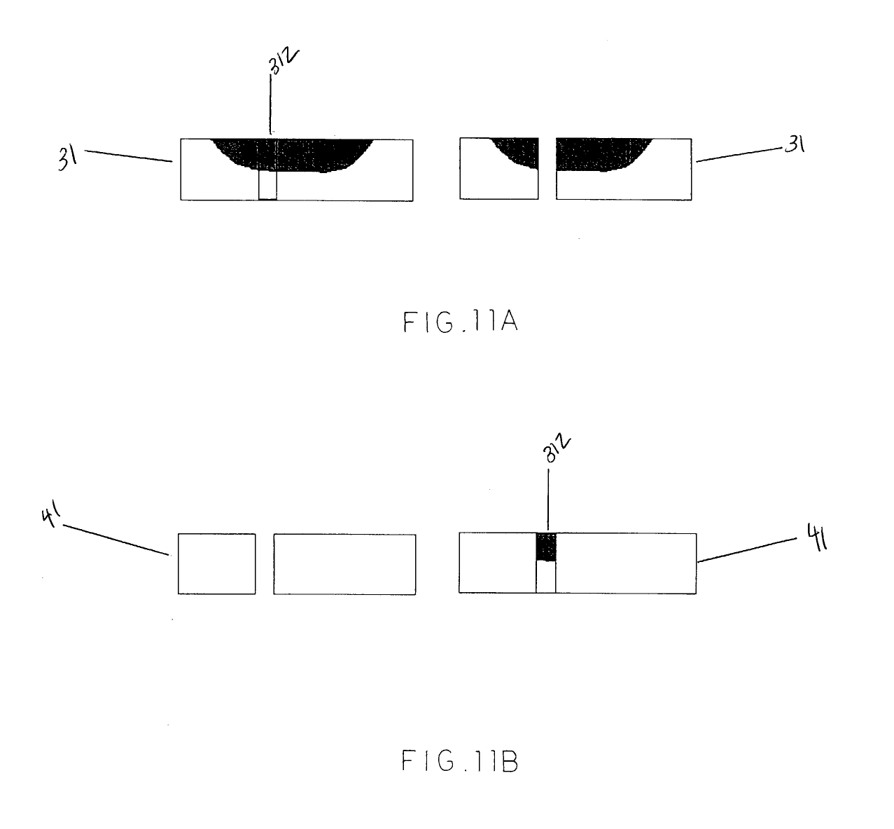

As shown in FIG. 9, in another iteration of the preferred process and preferred embodiment, disposable needles 321′ can be pre-loaded with donor tissue cores 312′ prior to use and deliver the pre-loaded donor tissue cores 312 into recipient arrays independent of the microscope station 2′ and the donor block holder 33′ and slide/donor platform 5′. A needle station is constructed of a recipient needle station 4′, combined with a recipient block mechanism 45′, together mounted on an independent deck plate, is used with pre-loaded donor punch needles 312′ to construct tissue arrays. In a preferred operation and embodiment, recipient block holes 411′ are punched and arranged in a grid pattern by means of a recipient needle punch 42′ and arrayed using the recipient X detent wheel 72′ and recipient Y detent wheel 73′ as described above. Alternatively, a recipient paraffin block 41′ containing pre-molded grid patterns can be locked into the recipient block holder 43′. Appropriate disposable donor punch needles 321′ with pre-loaded donor tissue cores 312′ are selected. A first pre-loaded disposable donor punch needle 321′ is locked into the donor needle collet 324′ of the independent needle station and extruded, as pushed by the stylus 326′, into the first selected recipient hole 411′ on the recipient paraffin block 41′. The exhausted pre-loaded disposable needle 321′ is removed from the collet 324′ and discarded and a second pre-loaded disposable needle 321″ is locked into the Collet 324′. The recipient block mechanism 43 is advanced by means of the X or Y or both X and Y Detent Wheels 73, 74, such that the second pre-loaded needle 321″ is now over the second hole in the pre-gridded pattern of the recipient paraffin block 41′. Again the pre-loaded needle 321′ is depressed just to the surface of the recipient paraffin block 41′ surface, the stylet 326′ is depressed to full extension and the second tissue core 312″ extruded into the second recipient block hole 411′. By repeating the process, customer arrays may be constructed with disposable pre-loaded needles and only a single recipient block mechanism 43.

The present instrument and different variations of the instant instrument offer many major technical and mechanical advances and improvements and economic benefits over the prior art. To summarize, the instant instrument and many variations thereof comprises, among many other unique features, an operatively linked microscope, with ocular reticule, to view, examine, align and index the reference donor slide with the donor tissue block for the purpose of precisely selecting tissue features and capturing them in cores through use of the donor needle punch; a separate holder for stained reference slide (sectioned from donor block and stained) operatively linked and indexed to donor block for precision punches; separate precision holders for donor block (under donor punch needle unit) and recipient block (under recipient punch needle unit); a recipient block indexed to a donor block; precision-indexed platform(s) to move recipient block, donor block, and reference slide in perfect alignments in reference one to another; precisely pitched stops or detents on the holder of the recipient block to exactly position holes for receiving the donor tissue cores for constructing the tissue arrays; independent needle/stylus assemblies that are disposable with the Needle and Stylus are of the same length for precise “Z” height delivery of donor tissue core into recipient block, the said “Z” height stops are adjustable for the separately operable donor needle-donor block, and recipient needle-recipient block; the said needle/stylus assemblies is easily assembled in a rotatable collet device for quick needle changes and breaking off tissue cores by rotation; independent or detachable recipient needle station with second detent for use with disposable needles; independent donor and recipient needle stations; disposable needles and disposable needles pre-loaded with cores for custom array assembly; and pre-made pre-gridded recipient block.

Having described the preferred embodiments of the present invention, it will appear to those ordinarily skilled in the art that various modifications, changes, adaptations, variations and modifications may be made to the disclosed embodiments without departing from the spirit of the present invention, and that such modifications are intended to be within the scope of the present invention. Accordingly, the invention is limited only by the following claims.

What is claimed is:

1. An instrument for obtaining a core from a donor tissue block, the instrument comprising: a donor tissue block station holding at least one donor tissue block; a reference slide station for holding a stained reference donor tissue slide sectioned from corresponding said donor tissue block; a microscope capable of viewing and indexing said stained reference donor tissue slide in said reference slide station; a punch needle station, comprising a donor punch needle and an associated stylet; and wherein said reference slide station and said donor tissue block station are operatively linked and the relative spatial position and location of said reference tissue slide is adjustable and indexed against said donor tissue block.

2. The instrument of claim 1, wherein said donor tissue block station and said reference slide station are operatively linked by a common platform.

3. The instrument of claim 2, wherein said common platform is moveable in at least one of an X and Y axis, whereby said common platform can be moved to place either said reference slide station or said donor tissue block station directly under the viewing field of said microscope.

4. The instrument of claim 1, wherein said microscope comprises a reticule with a center cross for indexing a microscopic viewing field.

5. The instrument of claim 4, wherein said microscope comprises an adjustable binocular head of said microscope, that can be adjusted in a set of X and Y directions, to center said ocular reticule to pinpoint a center of at least one donor tissue core region.

6. The instrument of claim 1, wherein said microscope comprises a lighting source for enhancing a microscopic viewing field.

7. The instrument of claim 1, wherein said reference slide station and said donor tissue block station is moveable along X- and Y-axis and said punch needle station is moveable along Z-axis.

8. The instrument of claim 1, wherein said reference slide station and said donor tissue block station are moveable in synchronization so that a center cross of a reticule of said microscope pinpoints to a donor core region and said donor punch needle pinpoints to said corresponding core region in said donor tissue block.

9. The instrument of claim 1, wherein said punch needle station further comprises a collet for holding said donor punch needle and said associated stylet as a punch needle assembly unit.

10. The punch needle station of claim 8, wherein said collet comprises a handle for rotating said punch needle assembly along Z axis for breaking off a tissue core.

11. The instrument of claim 1 further comprises a recipient punch needle station, comprising a punch needle, an associated stylet and optionally a collet with a handle for holding said punch needle and said associated stylet as a punch needle assembly unit.

12. The instrument of claim 11, wherein said collet comprises a handle and is rotatable by the said handle along a Z axis for breaking off a recipient block core to create a recipient hole.

13. The instrument of claim 11, wherein said recipient punch needle station is moveable along a Z axis.

14. The instrument of claim 1 further comprises a recipient block station holding at least one recipient block, wherein said recipient block station is moveable by linear screw drives or other mechanism with detents along the X and Y axis, whereby grids of a set of accurately spaced punched holes may be formed in said recipient block, with a predetermined distance between said punched holes corresponding to said distance stepped between individual detents.

15. The instrument of claim 14 further comprises a means for positioning said recipient block under said donor punch needle station, such that the alignment of said donor punch Z-axis and said recipient block is indexed to said last alignment between said recipient punch needle station and said recipient block.

16. The instrument of claim 1 further comprises:
a means by which a position of said donor punch needle station is indexed with respect to said recipient block of said recipient block station with a set of detent wheels; and
a means to set a stop or detent between said donor needle punch tip and said top surface of said recipient block with aid of a thumbwheel on said recipient block station.

17. An instrument for constructing an array of tissue cores, the instrument comprising:
a recipient block station holding at least one recipient block, comprising pre-gridded and pre-punched holes in said recipient block for receiving at least one donor tissue core from at least one pre-loaded donor punch needle, each having one of said donor tissue core;
a punch needle station, comprising a punch needle assembly capable of holding a disposable donor punch needle containing a pre-loaded tissue core and an associated stylet; and
wherein said donor punch needle station delivers a pre-loaded tissue core into said pre-punched and pre-gridded hole in said recipient block.

18. A punch needle station, comprising:
a punch needle;
an associated stylet;
a collet holding said punch needle and said associated stylet; and
wherein said collet is rotatable along a Z-axis to break off a punched tissue core from a tissue block.

19. The punch needle station of claim 18, wherein said stylet is moveable through said punch needle for passing through, a punch lumen of said punch needle.

20. The punch needle station of claim 18 further comprises:
a set of adjustable detents;
a spring;
a push button; and
wherein said punch needle is moveable along a Z-axis such that said punch needle's movement is controlled by said adjustable detents for limiting downward movement by said push button, by which a punching depth of said punch needle is precisely set, and upward movement by said spring for returning said punch needle to its said upward stop.

21. The punch needle station of claim 18, wherein said associated stylet further comprises a set of adjustable pre-set detents for limiting downward and upward movement, a spring for returning said stylet to its said upward stop, and a push button for downward movement.

22. The punch needle station of claim 18 wherein said punch needle station further comprises a donor needle station.

23. The punch needle station of claim 18 wherein said punch needle station further comprises a recipient needle station.

24. A disposable punch needle assembly, comprising:
a punch needle; and
an associated stylet.

25. The disposable punch needle assembly of claim 24, wherein said punch needle further comprises a pre-loaded tissue core.

26. The disposable punch needle assembly of claim 24, wherein said punch needle has an internal diameter ranging from 0.25 mm to 5 mm.

27. A pre-made recipient block with pre-punched holes in a pre-determined grid pattern for constructing a tissue array.

28. A process of constructing a tissue array block, comprising steps of:
a. microscopic viewing and selecting a desirable donor tissue core region on a reference donor tissue slide in a reference slide holder;
b. spatial indexing selected said desirable donor tissue core region to a source donor tissue block in a donor block holder in spatial relationship to a donor punch needle;
c. punching out said desirable donor tissue core in said source donor tissue block in said donor block holder by said donor punch needle;
wherein said microscopic viewing and selecting is achieved by using a microscope with a reticule with a center cross for viewing and indexing the features in the viewing field; and
wherein said spatial indexing is achieved by operatively linking said reference slide holder with said donor block holder and/or donor punch needle.

29. The process of claim 28 further comprising the steps of:
d. delivering said desirable donor tissue core into a hole in a recipient block.

30. The process of claim 28, wherein said reference donor tissue slide is a whole section from said source donor tissue block and stained immunohistologically or immunohistochemically.

31. The process of claim 28, wherein said operatively linking of said reference slide holder with said donor block holder and/or donor punch needle is achieved by a common platform for situating said reference slide holder and said donor block holder.

32. The process of claim 28, wherein said donor punch needle station comprises a donor punch needle and an associated stylet.

33. The process of claim 28, wherein said donor punch needle station further comprises a collet for holding said punch needle and said associated stylet, wherein said collet is rotatable along a Z-axis.

34. The process of claim 29, wherein said hole in said recipient block is punched out by a recipient punch needle station.

35. The process of claim 34, wherein said recipient punch needle station comprises a recipient punch needle and an associated stylet.

36. The process of claim 35 wherein said recipient punch needle station further comprises a collet for holding said recipient punch needle and said associated stylet, wherein said collet is rotatable along a Z-axis.

37. The process of claim 29, wherein said recipient block contains a set of pre-punched holes in a grid pattern.

38. The process of claim 29, further comprising the steps of having a>series of three or more Z-axis height adjustments, whereby the accuracy of>placement of tissue cores in recipient blocks is improved, by
d(1) operating a first Z-axis adjustment of said donor punch needle, whereby operating said donor punch needle against the said donor tissue block provides for said donor punch needle to extend precisely the depth of said donor tissue block, or some fixed partial depth thereof, and said Z-axis needle travel is fixed as a detent;
d(2) operating a second Z-axis adjustment of said recipient block, wherein said recipient block is placed under said donor punch needle, and a top surface of said recipient block is mated to the tip of the fully depressed said first Z-axis adjusted donor needle; and
d(3) operating a third Z-axis adjustment of said recipient punch needle, whereby at least one paraffin core of a thickness of said recipient block can be removed by means of depressing said recipient punch needle when alongside said recipient block, such that a tip of said recipient punch needle just touches a recipient block cassette, or some fixed partial depth thereof, such that said third Z-axis adjustment of said recipient punch needle is fixed as a detent, or is thereby adjusted for a partial depth by comparison to the depth of the donor block Z-axis adjustment.
